(19) United States
(12) Patent Application Publication
(10) Pub. No.: US 2024/0269107 A1
(43) Pub. Date: Aug. 15, 2024

(54) Methods of treating or reducing symptoms of brain cancer such as glioma and glioblastoma via combination therapy with allosteric modulators and phenethylamines.

(71) Applicant: David Alan Heldreth, JR., Bellevue, WA (US)

(72) Inventor: David Alan Heldreth, JR., Bellevue, WA (US)

(21) Appl. No.: 18/616,089

(22) Filed: Mar. 25, 2024

Related U.S. Application Data
(62) Division of application No. 17/667,147, filed on Feb. 8, 2022.
(60) Provisional application No. 63/207,183, filed on Feb. 10, 2021.

(51) Int. Cl. A61K 31/352; A61K 31/015; A61K 31/05; A61K 31/135; A61K 31/164; A61K 31/165; A61K 31/404; A61K 31/4045; A61K 31/473; A61K 31/4741; A61K 31/48; A61K 31/675; A61K 31/704; A61K 33/06; A61K 33/30; A61K 36/68; A61K 36/77

(52) U.S. Cl. CPC A61K 31/352; A61K 31/015; A61K 31/05; A61K 31/135; A61K 31/164; A61K 31/165; A61K 31/404; A61K 31/4045; A61K 31/473; A61K 31/4741; A61K 31/48; A61K 31/675; A61K 31/704; A61K 33/06; A61K 33/30; A61K 36/68; A61K 36/77

(57) ABSTRACT

The invention involves the use of formulations of positive, negative or other allosteric modulators of primarily 5ht2a receptors, but also those of: 5ht1a/b/c/d, 5ht2b/c, 5ht3, 5ht4, 5ht7, and other receptors in combination with phenethylamines and other compounds; to treat brain cancer such as glioma(s) and/or glioblastoma(s).

This patent (application) is a divisional patent filing which claims prior filing date/priority to patent, please reference patent title: Methods of use and formulations of allosteric modulators of the serotonin, dopamine and other receptor systems for medical, recreational, religious, research and other uses. Application Ser. No.: 17/667,147—which claims prior filing date to provisional patent. Please reference provisional patent title: Psychedelic formulations for medical, recreational, religious, research and other uses. Application No.: 63/207,183.

### FIELD OF THE INVENTION

The invention involves the use of formulations of positive, negative or other allosteric modulators of primarily 5ht2a receptors, but also those of: 5ht1a/b/c/d, 5ht2b/c, 5ht3, 5ht4, 5ht7, in combination with phenethylamines and other compounds; to treat brain cancer such as glioma(s) and/or glioblastoma(s).

### BACKGROUND OF THE INVENTION

People across the world are affected by brain cancers and the serotonin system is believed to be involved in many if not all of these conditions. Phenethylamines such as Mescaline, which is the active compound of many cacti, which crosses the blood brain barrier and stimulates 5-ht receptors and Phenethylamines compounds provide fast-acting and long-lasting changes to a person's illness.

However, there is variance in the effects these compounds have based on human genetics and related conditions. Allosteric binding occurs at a secondary binding site and not at the orthosteric or main agonist site and thus provides a way for additional compounds to be used in combination to provide ideal binding of receptors for individualized medicine and proper dosing. Allosteric binding affects the affinity and binding rate or activity of the receptor in a variety of ways including, but not limited to changing the shape of the receptor.

### SUMMARY OF THE INVENTION

The invention involves the use of formulations of positive, negative or other allosteric modulators of primarily 5ht2a receptors, but also those of: 5ht1a/b/c/d, 5ht2b/c, 5ht3, 5ht4, 5ht7, in combination with phenethylamines and other compounds; to treat brain cancer such as glioma(s) and/or glioblastoma(s).

### BRIEF DESCRIPTION OF THE DRAWINGS

PAGE 1 drawings show potential formulations of the compositions and the results of a human experiment on the use of proscaline alone or with positive 5HT2A allosteric modulators. PAGE 1 SHOWS FIG. 1 (FIG. 1) and FIG. 2 (FIG. 2).

FIG. 1 shows a recipe for a mescaline and THCV composition to be taken orally, but may be used in other ways.

FIG. 2. (FIG. 2) shows the results of a human experiment on the use of proscaline alone or with positive 5HT2A allosteric modulators.

Page 2 drawings show the results of a human experiment on the use of proscaline alone or with allosteric modulators.

FIG. 3. shows the results of a human experiment on the use of proscaline alone or with negative 5HT2A allosteric modulators.

FIG. 4. (FIG. 4) shows the results of a human experiment on the use of proscaline alone or with allosteric modulators to modulate fear, anxiety and nausea. The compounds used in addition to the psychedelics were: for Nausea: 5ht3 allosteric modulators: linalool, citral and ginger for nausea; for anxiety and fear: 5ht7 modulator zinc and 5ht1 modulator cannabidiol.

### DRAWING SUMMARY

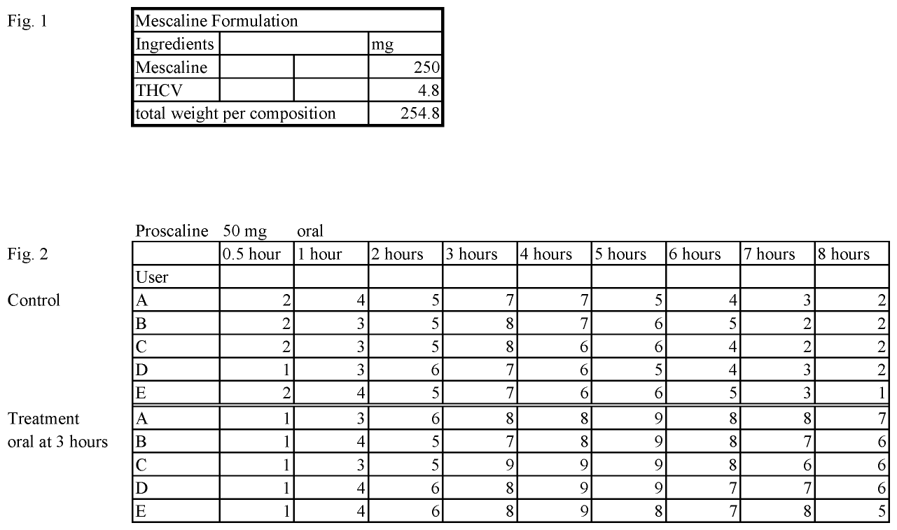

PAGE 1 drawings show potential formulations of the psychedelic compositions and the results of a human experiment on the use of proscaline alone or with positive 5HT2A allosteric modulators. PAGE 1 SHOWS FIG. 1 (FIG. 1) and FIG. 2 (FIG. 2). FIG. 1 shows a recipe for a mescaline and THCV composition to be taken orally, but may be used in other ways. FIG. 2. (FIG. 2) shows the results of a human experiment on the use of proscaline alone or with positive 5HT2A allosteric modulators.

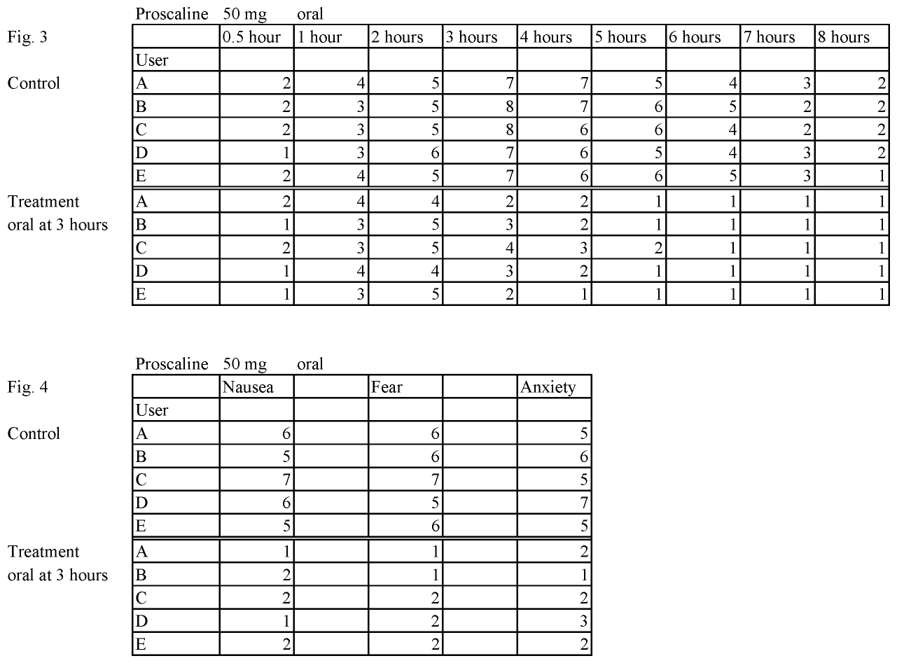

Page 2 drawings show the results of a human experiment on the use of proscaline alone or with allosteric modulators. FIG. 3. shows the results of a human experiment on the use of proscaline alone or with negative 5HT2A allosteric modulators. FIG. 4. (FIG. 4) shows the results of a human experiment on the use of proscaline alone or with allosteric modulators to modulate fear, anxiety and nausea. The compounds used in addition to the psychedelics were: for Nausea: 5ht3 allosteric modulators: linalool, citral and ginger for nausea; for anxiety and fear: 5ht7 modulator zinc and 5ht1 modulator cannabidiol.

### DETAILED DESCRIPTION OF THE INVENTION

The invention involves the use of formulations of positive, negative inverse agonist, or neutral allosteric modulators of primarily 5ht2 receptors, but also those of: 5ht1a/b/c/d, 5ht2a/b/c, 5ht3, 5ht4, 5ht5, 5ht6, 5ht7, dopamine receptors (D1, D2, D3, D4, D5), adrenergic receptors (α1A, α1B, α2A, α2B, α2C, β1, β2), serotonin transporter (SERT) in combination with phenethylamines, antifungals (including immune checkpoint inhibitors), vaccines and other items such as, but not limited to: cannabinoids, terpenes, flavonoids, minerals, compounds such as, but not limited to 5ht2a receptor agonists or other compounds; to treat brain cancer such as glioma(s) and/or glioblastoma(s).

Allosteric modulation is the manipulation of a receptor at a site other than normal binding site known as the orthosteric site. This is a less utilized method due to only recent discovery of its mechanisms as well as the need to identify these for each receptor. The use of allosteric modulators allows for precise alterations to the activity at the receptor and thus fine tuning of medical effects. Some embodiments utilize compounds which work as positive or negative allosteric modulators of the 5ht2a receptor or other 5ht systems. Allosteric modulation of 5ht2a also includes allosteric modulation through interaction with other receptor systems such as with heteromers or other such items.

In some embodiments the composition will include purified compounds which are either isolated or just purified. In other embodiments raw extracts or ground/processed biomass may be used. Some embodiments include excipients such as water, cyclodextrin, ethanol or other items off of the Food and Drug Administration authorized and approved excipient list.

Some embodiments will include utilizing nano technology, encapsulation, beta glucan particles, chitosan, yeast extract, surfactants, binders and other compounds to increase immune response, efficiency, availability, release lifespan, release speed among other parameters.

In some embodiments compositions eye drops, nasal spray, mouth spray, inhalers or other uses.

Some embodiments of the cancer treatment are used with vaccines, antibodies, cytokines, proteins, peptides, amino acids, DNA, RNA.

Potential compounds can be found below.

### Compound List

Phenethylamines including, but not limited to: mescaline, normescaline, 2c-i,2c-b, 2c-e. Includes all: phenethylamines in 2(X) series such as 2-CI including all NBOME, NBOH, and other analogs.

Beta carbolines and maoi inhibitors including, but not limited to: harmaline, tetraharmaline, norharmane, perlolyrine, tetrahydroharmine, harmane, harmine, harmol.

Antibiotics or antifungals such as penicillin, azithromycin or miconazole.
1 AEM alpha-Ethyl-3,4,5-trimethoxy-PEA
2 AL 4-Allyloxy-3,5-dimethoxy-PEA
3 ALEPH 4-Methylthio-2,5-dimethoxy-A
4 ALEPH-2 4-Ethylthio-2,5-dimethoxy-A
5 ALEPH-4 4-Isopropylthio-2,5-dimethoxy-A
6 ALEPH-6 4-Phenylthio-2,5-dimethoxy-A
7 ALEPH-7 4-Propylthio-2,5-dimethoxy-A
8 ARIADNE 2,5-Dimethoxy-alpha-ethyl-4-methyl-PEA
9 ASB 3,4-Diethoxy-5-methoxy-PEA
10 B 4-Butoxy-3,5-dimethoxy-PEA
11 BEATRICE 2,5-Dimethoxy-4,N-dimethyl-A
12 BIS-TOM 2,5-Bismethylthio-4-methyl-A
13 BOB 4-Bromo-2,5, beta-trimethoxy-PEA
14 BOD 2,5,beta-Trimethoxy-4-methyl-PEA
15 BOH beta-Methoxy-3,4-methylenedioxy-PEA
16 BOHD 2,5-Dimethoxy-beta-hydroxy-4-methyl-PEA
17 BOM 3,4,5, beta-Tetram ethoxy-P E A
18 4-Br-3,5-DMA 4-Bromo-3,5-dimethoxy-A
19 2-Br-4,5-MDA 2-Bromo-4,5-methylenedioxy-A
20 2C-B 4-Bromo-2,5-dimethoxy-PEA
21 3C-BZ 4-Benzyloxy-3,5-dimethoxy-A
22 2C-C 4-Chloro-2,5-dimethoxy-PEA
23 2C-D 4-Methyl-2,5-dimethoxy-PEA
24 2C-E 4-Ethyl-2,5-dimethoxy-PEA
25 3C-E 4-Ethoxy-3,5-dimethoxy-A
26 2C-F 4-Fluoro-2,5-dimethoxy-PEA
27 2C-G 3,4-Dimethyl-2,5-dimethoxy-PEA
28 2C-G-3 3,4-Trimethylene-2,5-dimethoxy-PEA
29 2C-G-4 3,4-Tetramethylene-2,5-dimethoxy-PEA
30 2C-G-5 3,4-Norbornyl-2,5-dimethoxy-PEA
31 2C-G-N 1,4-Dimethoxynaphthyl-2-ethylamine
32 2C-H 2,5-Dimethoxy-PEA
33 2C-I 4-lodo-2,5-dimethoxy-PEA
34 2C-N 4-Nitro-2,5-dimethoxy-PEA
35 2C-O-4 4-Isopropoxy-2,5-dimethoxy-PEA
36 2C-P 4-Propyl-2,5-dimethoxy-PEA
37 CPM 4-Cyclopropylmethoxy-3,5-dimethoxy-PEA
38 2C-SE 4-Methylseleno-2,5-dimethoxy-PEA
39 2C-T 4-Methylthio-2,5-dimethoxy-PEA
40 2C-T-2 4-Ethylthio-2,5-dimethoxy-PEA
41 2C-T-4 4-Isopropylthio-2,5-dimethoxy-PEA
42 psi-2C-T-4 4-Isopropylthio-2,6-dimethoxy-PEA
43 2C-T-7 4-Propylthio-2,5-dimethoxy-PEA
44 2C-T-8 4-Cyclopropylmethylthio-2,5-dimethoxy-PEA
45 2C-T-9 4-(t)-Butylthio-2,5-dimethoxy-PEA
46 2C-T-13 4-(2-Methoxyethylthio)-2,5-dimethoxy-PEA
47 2C-T-15 4-Cyclopropylthio-2,5-dimethoxy-PEA
48 2C-T-17 4-(s)-Butylthio-2,5-dimethoxy-PEA
49 2C-T-21 4-(2-Fluoroethylthio)-2,5-dimethoxy-PEA
50 4-D 4-Trideuteromethyl-3,5-dimethoxy-PEA
51 beta-D beta,beta-Dideutero-3,4,5-trimethoxy-PEA
52 DESOXY 4-Methyl-3,5-Dimethoxy-PEA
53 2,4-DMA 2,4-Dimethoxy-A
54 2,5-DMA 2,5-Dimethoxy-A
55 3,4-DMA 3,4-Dimethoxy-A
56 DMCPA 2-(2,5-Dimethoxy-4-methylphenyl)-cyclopropylamine
57 DME 3,4-Dimethoxy-beta-hydroxy-PEA
58 DMMDA 2,5-Dimethoxy-3,4-methylenedioxy-A
59 DMMDA-2 2,3-Dimethoxy-4,5-methylenedioxy-A
60 DMPEA 3,4-Dimethoxy-PEA
61 DOAM 4-Amyl-2,5-dimethoxy-A
62 DOB 4-Bromo-2,5-dimethoxy-A
63 DOBU 4-Butyl-2,5-dimethoxy-A
64 DOC 4-Chloro-2,5-dimethoxy-A
65 DOEF 4-(2-Fluoroethyl)-2,5-dimethoxy-A
66 DOET 4-Ethyl-2,5-dimethoxy-A
67 DOI 4-lodo-2,5-dimethoxy-A
68 DOM (STP) 4-Methyl-2,5-dimethoxy-A
69 psi-DOM 4-Methyl-2,6-dimethoxy-A
70 DON 4-Nitro-2,5-dimethoxy-A
71 DOPR 4-Propyl-2,5-dimethoxy-A
72 E 4-Ethoxy-3,5-dimethoxy-PEA
73 EEE 2,4,5-Triethoxy-A
74 EEM 2,4-Diethoxy-5-methoxy-A
75 EME 2,5-Diethoxy-4-methoxy-A
76 EMM 2-Ethoxy-4,5-dimethoxy-A
77 ETHYL-J N, alpha-diethyl-3,4-methylenedioxy-PEA
78 ETHYL-K N-Ethyl-alpha-propyl-3,4-methylenedioxy-PEA
79 F-2 Benzofuran-2-methyl-5-methoxy-6-(2-aminopropane)
80 F-22 Benzofuran-2,2-dimethyl-5-methoxy-6-(2-aminoprop ane)
81 FLEA N-Hydroxy-N-methyl-3,4-methylenedioxy-A
82 G-3 3,4-Trimethylene-2,5-dimethoxy-A
83 G-4 3,4-Tetramethylene-2,5-dimethoxy-A
84 G-5 3,4-Norbornyl-2,5-dimethoxy-A
85 GANESHA 3,4-Dimethyl-2,5-dimethoxy-A
86 G-N 1,4-Dimethoxynaphthyl-2-isopropylamine
87 HOT-2 2,5-Dimethoxy-N-hydroxy-4-ethylthio-PEA
88 HOT-7 2,5-Dimethoxy-N-hydroxy-4-(n)-propylthio-PEA
89 HOT-17 2,5-Dimethoxy-N-hydroxy-4-(s)-butylthio-PEA
90 IDNNA 2,5-Dimethoxy-N,N-dimethyl-4-iodo-A
91 IM 2,3,4-Trimethoxy-PEA
92 IP 3,5-Dimethoxy-4-isopropoxy-PEA
93 IRIS 5-Ethoxy-2-methoxy-4-methyl-A
94 J alpha-Ethyl-3,4-methylenedioxy-PEA
95 LOPHOPHINE 3-Methoxy-4,5-methylenedioxy-PEA
96 M 3,4,5-Trimethoxy-PEA
97 4-MA 4-Methoxy-A
98 MADAM-6 2,N-Dimethyl-4,5-methylenedioxy-A
99 MAL 3,5-Dimethoxy-4-methallyloxy-PEA
100 MDA 3,4-Methylenedioxy-A
101 MDAL N-Allyl-3,4-methylenedioxy-A
102 MDBU N-Butyl-3,4-methylenedioxy-A
103 MDBZ N-Benzyl-3,4-methylenedioxy-A
104 MDCPM N-Cyclopropylmethyl-3,4-methylenedioxy-A
105 MDDM N,N-Dimethyl-3,4-methylenedioxy-A
106 MDE N-Ethyl-3,4-methylenedioxy-A
107 MDHOET N-(2-Hydroxyethyl)-3,4-methylenedioxy-A
108 MDIP N-Isopropyl-3,4-methylenedioxy-A
109 MDMA N-Methyl-3,4-methylenedioxy-A
110 MDMC N-Methyl-3,4-ethylenedioxy-A
111 MDMEO N-Methoxy-3,4-methylenedioxy-A
112 MDMEOET N-(2-Methoxyethyl)-3,4-methylenedioxy-A
113 MDMP alpha,alpha, N-Trimethyl-3,4-methylenedioxy-PEA
114 MDOH N-Hydroxy-3,4-methylenedioxy-A
115 MDPEA 3,4-Methylenedioxy-PEA
116 MDPH alpha,alpha-Dimethyl-3,4-methylenedioxy-PEA
117 MDPL N-Propargyl-3,4-methylenedioxy-A
118 MDPR N-Propyl-3,4-methylenedioxy-A
119 ME 3,4-Dimethoxy-5-ethoxy-PEA
120 MEDA 3-methoxy-4,5-Ethylenedioxy-A [Erowid corrected]
121 MEE 2-Methoxy-4,5-diethoxy-A
122 MEM 2,5-Dimethoxy-4-ethoxy-A
123 MEPEA 3-Methoxy-4-ethoxy-PEA
124 META-DOB 5-Bromo-2,4-dimethoxy-A
125 META-DOT 5-Methylthio-2,4-dimethoxy-A
126 METHYL-DMA N-Methyl-2,5-dimethoxy-A
127 METHYL-DOB 4-Bromo-2,5-dimethoxy-N-methyl-A
128 METHYL-J N-Methyl-alpha-ethyl-3,4-methylenedioxy-PEA
129 METHYL-K N-Methyl-alpha-propyl-3,4-methylenedioxy-PEA
130 METHYL-MA N-Methyl-4-methoxy-A
131 METHYL-MMDA-2 N-Methyl-2-methoxy-4,5-methylenedioxy-A
132 MMDA 3-Methoxy-4,5-methylenedioxy-A
133 MMDA-2 2-Methoxy-4,5-methylenedioxy-A
134 MMDA-3a 2-Methoxy-3,4-methylenedioxy-A
135 MMDA-3b 4-Methoxy-2,3-methylenedioxy-A
136 MME 2,4-Dimethoxy-5-ethoxy-A
137 MP 3,4-Dimethoxy-5-propoxy-PEA
138 MPM 2,5-Dimethoxy-4-propoxy-A
139 ORTHO-DOT 2-Methylthio-4,5-dimethoxy-A
140 P 3,5-Dimethoxy-4-propoxy-PEA
141 PE 3,5-Dimethoxy-4-phenethyloxy-PEA
142 PEA PEA
143 PROPYNYL 4-Propynyloxy-3,5-dimethoxy-PEA
144 SB 3,5-Diethoxy-4-methoxy-PEA
145 TA 2,3,4,5-Tetramethoxy-A
146 3-TASB 4-Ethoxy-3-ethylthio-5-methoxy-PEA
172 5-TOM 2-Methoxy-4-methyl-5-methylthio-A
173 TOMSO 2-Methoxy-4-methyl-5-methylsulfinyl-A
174 TP 4-Propylthio-3,5-dimethoxy-PEA
175 TRIS 3,4,5-Triethoxy-PEA
176 3-TSB 3-Ethoxy-5-ethylthio-4-methoxy-PEA
177 4-TSB 3,5-Diethoxy-4-methylthio-PEA
178 3-T-TRIS 4,5-Diethoxy-3-ethylthio-PEA
179 4-T-TRIS 3,5-Diethoxy-4-ethylthio-PEA

Additionally includes all: (4-acetoxy) (4-hydroxy)(dimethyl) (diethyl) (N-methyl-N-ethyl) (N-methyl)(N-methyl-N-isopropyl)(N,N-diisopropyl) variations of compounds in compound list.

Additionally includes all: functional group variants, fumerates, fumerics, iodines, hydrofumarates, deneutered or not, salts, acids, isomers, analogs, precursors, further metabolites of biosynthetic or synthetic pathways.

Also: Zinc, Magnesium, Sulfate, Carvelidol.

1. The method of treating or reducing symptoms (tumor size, tumor spread, etc) of brain cancers such as glioblastoma, glioma, and other forms/types of brain cancer or cancers in the brain, consisting of:
Administering to a human patient a therapeutic amount of a composition comprising:
a 5ht receptor allosteric modulator or modulators, selected from: THCV (Tetrahydrocannabivarin), oleamide, linalool, limonene, tetrahydrocannabinol, cannabidiol or zinc;
AND one item or a mixture of: mescaline, hordenine, anhalonidine, pellotine or other phenethylamine; AND
Administering to a human patient a therapeutic amount of an antifungal composition comprising either: azithromycin, Panobinostat, temozolamide, miconazole, Nivolumab, Pembrolizumab, Atezolizumab, Avelumab, Durvalumab, Cemiplimab, Dostarlimab, Retifanlimab, Toripalimab, doxorubicin, or a mixture thereof or multiple compounds sequentially; AND OPTIONALLY
administering to a human patient a therapeutic amount of a composition comprising: A 5ht, allosteric modulator or modulators selected from: palmitoleamide, 2,2-dimethyloleamide, N-oleoyl glycine, myristoleamide, 1-oleyl-2-acetylglycerol, anandamide, oleyl aldehyde, trifluoromethyl ketone, oleic acid, oleylpropanolamide alpha-pinene, cannabidiol, carvelidol.

2. The method of claim 1 including testing/diagnosing the human patient such as, but not limited to: cancer biomarkers and genetic testing.

3. The method of claim 1 including administering to a human patient a therapeutic amount of a composition comprising either: harmine, hamaline, or other harmala alkaloids or a combination thereof.

4. The method of claim 1 including administering to a human patient a therapeutic amount of a composition comprising either: valencene, borneol, alpha-pinene, limonene, linalool, another terpene/terpenoid or a mixture thereof.

5. The method of claim 1 including administering to a human patient a therapeutic amount of a composition comprising: a vaccine or antibodies which target brain cancer, gliomas or glioblastoma.

6. The method of claim 1 including a step of administering to a human patient a therapeutic amount of: an allosteric modulator of the 5ht3, 5ht4, or 5ht7 receptors.

7. The method of claim 1 wherein the method of administering the treatment to a human patient follows the 21-day protocol which repeats until remission:
Administer to a human patient: 10-500 mg borneol, 5-50 mg panobinostat once day every other day for 14 days, followed by 7 days off.
During 7-day off cycle from Panobinostat, administer to a human patient: 10-500 mg borneol, 10-500 mg linalool, 10-200 mg mescaline, and 1-50 mg hordenine. Followed by an OPTIONAL same-day treatment of 1-5 mg of carvelidol or 50-500 mg of alpha-pinene.

8. The method of claim 1 wherein the method of administering the treatment to a human patient follows the 21-day protocol which repeats until remission:
Administer to a human patient: 10-500 mg borneol, AND administer to a human patient doxorubicin (20-75 milligrams (mg) per square meter of body size once every 21 days).
During the 20-days off from doxorubicin, administer to a human patient: 10-500 mg borneol, 10-500 mg linalool, 10-200 mg mescaline, 1-50 mg hordenine. Followed by an OPTIONAL same-day treatment of 1-5 mg of carvelidol or 50-500 mg of alpha-pinene.

9. The method of claim 1 wherein the method of administering the treatment to a human patient follows the 21-day protocol which repeats until remission:
Administer to a human patient: 10-500 mg borneol, AND administer to a human patient Durvalumab, 10-50 mg/kg, once every 21 days.
During the 20-days off from Durvalumab, administer to a human patient: 10-500 mg borneol, 10-500 mg linalool, 10-200 mg mescaline, 1-50 mg hordenine. Followed by an OPTIONAL same-day treatment of 1-5 mg of carvelidol or 50-500 mg of alpha-pinene.

10. The method of claim 1 wherein the method of administering the treatment to a human patient follows the 21-day protocol which repeats until remission:
Administer to a human patient: 10-500 mg borneol, doxorubicin (20-75 mg per square meter of body size), and Pembrolizumab (10-200 mg/kg), once every 21 days.
During the 20-days off from doxorubicin/durvalumab, administer to a human patient: 10-500 mg borneol, 10-500 mg linalool, 10-200 mg mescaline, 1-50 mg hordenine. Followed by an OPTIONAL same-day treatment of 1-5 mg of carvelidol or 10-500 mg of alpha-pinene.

11. The method of treating or reducing symptoms of brain cancers such as glioblastoma, glioma, and other forms/types of brain cancer or cancers in the brain, consisting of:
Administering to a human patient a therapeutic amount of a composition comprising:
a 5ht receptor, allosteric modulator or modulators, selected from: THCV (Tetrahydrocannabivarin), oleamide, linalool, limonene, tetrahydrocannabinol, cannabidiol or zinc;
AND one item or a mixture of: proscaline (4-propoxy-3,5-dimethoxyphenethylamine or 4-propoxy-3,5-DMPEA), N-methylmescaline, N,N-Diformylmescaline, methylmescaline, normescaline, n-acetylmescaline, 2C-B, 2C-C, 2C-D, 2C-E, 2C-I, 2C-P, 2C-T, 2C-T2, 2C-T4, 2C-T7, 2C-T9, Trimethoxyamphetamine, or 3,4-Methylenedioxy methamphetamine; AND
Administering to a human patient a therapeutic amount of an antifungal composition comprising either: azithromycin, Panobinostat, temozolamide, miconazole, Nivolumab, Pembrolizumab, Atezolizumab, Avelumab, Durvalumab, Cemiplimab, Dostarlimab, Retifanlimab, Toripalimab, doxorubicin, or a mixture thereof or multiple compounds sequentially; AND OPTIONALLY
administering to a human patient a therapeutic amount of a composition comprising: A 5ht receptor allosteric modulator or modulators selected from: palmitoleamide, 2,2-dimethyloleamide, N-oleoyl glycine, myristoleamide, 1-oleyl-2-acetylglycerol, anandamide, oleyl aldehyde, trifluoromethyl ketone, oleic acid, oleylpropanolamide alpha-pinene, cannabidiol, carvelidol.

12. The method of claim 11 including testing/diagnosing the human patient such as, but not limited to: cancer biomarkers and genetic testing.

13. The method of claim 11 including administering to a human patient a therapeutic amount of a composition comprising either: harmine, hamaline, or other harmala alkaloids or a combination thereof.

14. The method of claim 11 including administering to a human patient a therapeutic amount of a composition comprising either: valencene, borneol, alpha-pinene, linalool, limonene or a mixture thereof.

15. The method of claim 11 including administering to a human patient a therapeutic amount of a composition comprising: a vaccine or antibodies which target brain cancer, gliomas or glioblastoma.

16. The method of claim 11 including a step of administering to a human patient a therapeutic amount of: an allosteric modulator of the 5ht3, 5ht4, or 5ht7 receptors.

17. The method of claim 11 wherein the method of administering the treatment to a human patient follows the 21-day protocol which repeats until remission:
Administer to a human patient: 10-500 mg borneol, 5-50 mg panobinostat once day every other day for 14 days, followed by 7 days off.
During 7-day off cycle from Panobinostat, administer to a human patient: 10-500 mg borneol, 10-500 mg linalool, 10-100 mg proscaline. Followed by an OPTIONAL same-day treatment of 1-5 mg of carvelidol or 50-500 mg of alpha-pinene.

18. The method of claim 11 wherein the method of administering the treatment to a human patient follows the 21-day protocol which repeats until remission:
Administer to a human patient: 10-500 mg borneol, AND administer to a human patient doxorubicin (20-75 milligrams (mg) per square meter of body size once every 21 days).
During the 20-days off from doxorubicin, administer to a human patient: 10-500 mg borneol, 10-500 mg linalool, 10-100 mg proscaline. Followed by an OPTIONAL same-day treatment of 1-5 mg of carvelidol or 50-500 mg of alpha-pinene.

19. The method of claim 11 wherein the method of administering the treatment to a human patient follows the 21-day protocol which repeats until remission:
Administer to a human patient: 10-500 mg borneol, AND administer to a human patient Durvalumab, 10-50 mg/kg, once every 21 days.
During the 20-days off from Durvalumab, administer to a human patient: 10-500 mg borneol, 10-500 mg linalool, 10-200 mg Trimethoxyamphetamine. Followed by an OPTIONAL same-day treatment of 1-5 mg of carvelidol or 50-500 mg of alpha-pinene.

20. The method of claim 11 wherein the method of administering the treatment to a human patient follows the 21-day protocol which repeats until remission:
Administer to a human patient: 10-500 mg borneol, 5-50 mg panobinostat once day every other day for 14 days, followed by 7 days off.
Administer to a human patient: 10-500 mg borneol, Pembrolizumab (10-200 mg/kg), once every 21 days.
During a day which is during the 20-days off from durvalumab, and the 7-days off from Panobinostat, administer to a human patient: 10-500 mg borneol, 10-500 mg linalool, 10-100 mg 2C-I. Followed by an OPTIONAL same-day treatment of 1-5 mg of carvelidol or 10-500 mg of alpha-pinene.
